(19) United States
(12) Patent Application Publication
(10) Pub. No.: US 2013/0254991 A1
(43) Pub. Date: Oct. 3, 2013

(54) PATIENT SUPPORT PLATFORM SYSTEM FOR BREAST THERAPY

(71) Applicant: Charles Y. Shang, Boynton Beach, FL (US)

(72) Inventor: Charles Y. Shang, Boynton Beach, FL (US)

(21) Appl. No.: 13/706,284

(22) Filed: Dec. 5, 2012

Related U.S. Application Data
(60) Provisional application No. 61/566,880, filed on Dec. 5, 2011.

(51) Int. Cl. A61B 6/04

(52) U.S. Cl. CPC A61B 6/0435
USPC 5/601

(57) ABSTRACT

A lateral decubitus precision patient support system and method for accurately and reproducibly positioning a patient's breast relative to a patient support surface to receive therapy, such as, but not limited to, radiation treatment, while the patient is in a lateral decubitus position (lying on the patient's side) upon a patient support surface is disclosed. The lateral decubitus precision support assembly may be securely attached to a treatment table during use. The lateral decubitus precision support assembly may include a contralateral breast module having at least one contralateral breast receiving area into which a contralateral breast of the patient may reside during breast therapy. The lateral decubitus precision support assembly may also include a treatment breast support that contacts a treatment breast of the patient to separate the treatment breast from the contralateral breast of patient to limit exposure of the contralateral breast to therapeutic procedures administered to the patient.

### CROSS-REFERENCE TO RELATED APPLICATION

This application claims priority to U.S. Provisional Patent Application No. 61/566,880, filed Dec. 5, 2011, the entirety of which is incorporated herein.

### FIELD OF THE INVENTION

The invention relates to medical patient support systems, and more particularly, to a patient support systems configured to support patients undergoing radiation therapy requiring accurate repeatability.

### BACKGROUND OF THE INVENTION

In the United States, breast cancer has been a very common cancer among women. In fact, the American Cancer Society has estimated that over 230,000 breast cancer cases exist in women in the United States in 2012. Despite of advancements in cancer treatment, it is further estimated that about 40,000 of these breast cancer cases may result in fatalities. To improve the survival rate, a generally accepted methods for treating breast cancer is a lumpectomy followed by uniform dosage of external radiation treatment throughout the breast plus margins to eliminate potential remaining live cancer cells following a surgical removal through the lumpectomy. The success of this treatment relies, in part, on the degree of uniformity of delivery of the radiation dose to the treated breast tissue. In addition, during application of the treatment, it is highly desirable to minimize the radiation received by surrounding organs, such as contra-lateral breast, lungs, and the heart to limit the detrimental effects of radiation to these organs.

Patients treated with external radiation therapy often experience varying side effects from the radiation treatments. In particular, those patients with large, pendulous or irregular shaped breasts are known to be particularly susceptible to undesirable cosmetic occurrences on their skin and to increased toxicities to the skin, the lungs and other body structures from the radiation treatments. Furthermore, patients have been more likely to experience toxicities to the heart when the radiation beams were unable to clear from the heart in those patients who have undergone left breast radiation treatment.

External radiation treatments for breast cancer are typically carried out with the patient in the supine (face-up) position and sometimes in the prone (face-down) position. Both positions demonstrated certain advantages and disadvantages. While the supine position is considered more reproducible for providing treatment, particularly with respect to women with large or irregularly shaped breasts, this position results in greater irradiation to surrounding areas in patients, such as, the lungs and heart, and requires the use of less homogeneous dose distributions. Because of the lateral displacement of breast tissue over the anterior chest wall that occurs when a patient is in supine position, a large separation is created that results in a non-uniform dose distribution, and large portions of the heart and adjacent lung are irradiated. In addition, the patient's respiration motion in the supine position greatly increases the uncertainty of the geometric and radiation doses for anterior chest wall, breasts, heart and lungs. Attempts have been made to minimize these problems, especially in women with large or irregularly shaped breasts, by modifying traditional treatment techniques such as through the use of breast holding bra or ring, breath-holding technique, wedge filters, high-energy photon beams, bolus, and intensity modulated beams, with limited success.

When a prone technique is used with a women having large or irregularly shaped breasts, the breast tissue is stretched downward thereby reducing the thickness of the breast and enabling a more homogeneous dose distribution than is often possible using the supine position. Nonetheless, the prone technique requires meticulous patient positioning and active patient participation to be successful. The prone technique often fails when used with less mobile patients, such as geriatric patients, weak patients, those patients with shoulder problems or patients with other issues caused by recent breast surgery. In addition, the prone technique also increases the difficulty in matching supraclavical and other lymph nodal treatment fields due to poor visibility of the treatment field projections. Moreover, the prone technique moves the heart closer to the anterior chest wall, which increases the difficulty of sparing a patient's heart of exposure to radiation fields. Furthermore, repeatability of the patient's position during therapy is often very difficult because such accuracy relies heavily on the patient's cooperation during setup in the prone position. In reality, only a small number of patients may be suitable for using the prone technique, particularly after breast surgery and chemotherapy.

### SUMMARY OF THE INVENTION

A lateral decubitus precision patient support system and method of using the system for accurately and reproducibly positioning a patient's breast relative to a patient support surface to receive therapy, such as, but not limited to, radiation treatment, while the patient is in a lateral decubitus position (lying on the patient's side) upon the patient support surface is disclosed. The lateral decubitus precision patient support system may enable a patient's upper breast to be accurately positioned at a horizontal and forward extending position for being irradiated or imaged, while the contralateral breast, arms, lungs and the heart are spared further or shielded completely from treatment radiation beams. The lateral decubitus precision patient support system may be securely attached to a treatment table during use. The treatment table may be a table furnished in an existing therapy unit or radiological imaging unit. The lateral decubitus precision patient support system may include a contralateral breast module having at least one contralateral breast receiving area into which a contralateral breast of the patient may reside during breast therapy to somewhat shield the contralateral breast from exposure and ultimately side effects of that exposure. The lateral decubitus precision patient support system may also include a treatment breast support that contacts a treatment breast of the patient to separate the treatment breast from the contralateral breast of the patient to limit exposure of the contralateral breast to therapeutic procedures administered to the patient.

The lateral decubitus precision patient support system may be usable to create a reproducible lateral decubitus position with an effectively separated contralateral breast technique that can be used to overcome the limitations of treating patients with large breasts, irregularly shaped breasts or pendulum breasts; to increase accuracy for regular breast radiation treatment; to decrease the irradiation to the heart; to reduce the radiation toxicity to the lungs; and to improve the cosmetic results due to lowered skin radiation doses. Treating a patient positioned in a lateral decubitus position yields the advantage demonstrated in the prone technique in expanding the treatment breast to a reduced thickness in the radiation beam angles resulting in more homogeneous dose distribution and less skin dose; yields the advantage shown in the supine setup in its undemanding process, superior visibility for treatment areas including adjacent lymphatic nodal areas, and open gap between the head from the anterior chest wall and irradiated field. Unlike previous techniques, the method of using the lateral decubitus precision patient support system, referred to as the lateral decubitus technique described herein, clearly promotes the separation of the contralateral breast from the treatment breast and radiation field, which may further reduce the possibility of a secondary cancer forming in the healthy, contralateral breast. In contrast to conventional techniques, use of the lateral decubitus precision patient support system may expand the lung on the treatment side towards the non-treatment side by gravity, thereby leading to lower lung toxicity. Use of the lateral decubitus precision patient support system may also enable more freedom for a patients' arms. For example, in addition to the common practice of holding a lesion side arm above a patient's head to avoid being irradiated, the lesion side arm may also be placed behind the patient's back without being irradiated, which may create more stable and reproducible setups, especially for the patients with limited or difficult arm motion. The lateral decubitus precision patient support system overcomes the disadvantages of conventional systems supine and prone patient positions. The lateral decubitus precision patient support system allows a patient to be comfortably, rapidly, reproducibly positioned for purposes of radiation treatment or therapy and also allows irradiation of almost all breast sizes from multiple beam projectiles, including radiation arcs.

The lateral decubitus precision patient support system for breast therapy may include one or more patient support surfaces configured to support a patient in a lateral decubitus position on the patient support surface. The lateral decubitus precision patient support system may also include one or more contralateral breast modules having at least one contralateral breast receiving area into which a contralateral breast of patient resides during breast therapy. The contralateral breast receiving area of the contralateral breast module may be positioned within the patient support surface. The contralateral breast module may also include a removable contralateral breast support configured to at least partially fill the contralateral breast receiving area in the contralateral breast module and to support the contralateral breast of patient before breast therapy.

The lateral decubitus precision patient support system may also include treatment breast support that contacts a treatment breast of a patient to separate the treatment breast from the contralateral breast of patient to limit exposure of the contralateral breast to therapeutic procedures administered to the patient. The treatment breast support may also include at least one lower lifting support extending from a treatment breast support body and configured to support the treatment breast of patient at a higher position relative to the patient support surface when the patient is in the lateral decubitus position than the treatment breast would naturally be positioned when the patient is in the lateral decubitus position. The treatment breast support may also include at least one upper lifting support that is adjustable relative to the lower lifting support such that a treatment breast is held in a forward extended, reproducible position for therapy on the treatment breast. A contact surface of the lower lifting support of the treatment breast support that contacts the treatment breast of the patient may be formed from a deformable material to comfort the treatment breast of the patient. At least a portion of the lower lifting support that contacts the treatment breast of the patient may be flexible to readily conform to the shape of the treatment breast to comfort the treatment breast of the patient. The lower lifting support may extend from the treatment breast support body laterally away from the treatment breast support body and away from a patient receiving area on the patient support surface.

The lateral decubitus precision patient support system may include a number of components useful to increase the repeatability of the position of the treatment breast and to increase the comfort of the patient while the patient is lying on the patient support surface of the support body. In particular, the lateral decubitus precision patient support system may include an adjustable abdominal support for maintaining position of a torso of the patient relative to the at least one patient support surface, and thereby maintain position of a treatment breast, in a repeatable position. The lateral decubitus precision patient support system may include an adjustable back support for maintaining position of a torso of the patient relative to the at least one patient support surface, and thereby maintain position of a treatment breast, in a repeatable position. The lateral decubitus precision patient support system may include a head support for maintaining position of a head of the patient relative to the at least one patient support surface, and thereby maintain position of a treatment breast, in a repeatable position. The head support may be adjustable such that the head contact surface is adjustable vertically upward relative to the patient support surface. The lateral decubitus precision patient support system may also include an adjustable arm support for supporting a lesion side arm of the patient, wherein the adjustable arm support is rotatable about a pivot point on the at least one patient support surface. Adjustable handles may also be provided for gripping by a patient's hands to provide stability and comfort to the patient and to create a reproducible position of a treatment breast.

The lateral decubitus precision patient support system may include a table engagement system having at least one connector for attaching the patient support surface to a table. The lateral decubitus precision patient support system may also include a breast holder device configured to bind the contralateral breast of the patient to the patient's body while pushing the contralateral breast away from treatment fields directed at a treatment breast of the patient.

These and other embodiments are described in more detail below.

### BRIEF DESCRIPTION OF THE DRAWING

The accompanying drawings, which are incorporated in and form a part of the specification, illustrate embodiments of the presently disclosed invention and, together with the description, disclose the principles of the invention.

FIG. 1 is a top lateral oblique perspective view of a lateral decubitus precision patient support system.

FIG. 2 is a bottom superior oblique perspective view of the lateral decubitus precision patient support system.

FIG. 3 is a superior view of the lateral decubitus precision patient support system.

FIG. 4 is an inferior view of the lateral decubitus precision patient support system.

FIG. 5 is a top view of the lateral decubitus precision patient support system.

FIG. 6 is a bottom view of the lateral decubitus precision patient support system.

FIG. 7 is a left side view of the lateral decubitus precision patient support system.

FIG. 8 is a right side view of the lateral decubitus precision patient support system.

FIG. 9 is a left side view of the lateral decubitus precision patient support system with some components removed to assist in viewing the components.

FIG. 10 is a superior side view of the lateral decubitus precision patient support system with some components removed to assist in viewing the components.

FIG. 11 is a right side view of the lateral decubitus precision patient support system with some components removed to assist in viewing the components.

FIG. 12 is an inferior view of the lateral decubitus precision patient support system with some components removed to assist in viewing the components.

FIG. 13 is a top view of the lateral decubitus precision patient support system with some components removed to assist in viewing the components.

FIG. 14 is a bottom view of the lateral decubitus precision patient support system with some components removed to assist in viewing the components.

FIG. 15 is a top lateral oblique perspective view of the lateral decubitus precision patient support system with some components removed to assist in viewing the components.

FIG. 16 is a bottom lateral oblique perspective view of the lateral decubitus precision patient support system with some components removed to assist in viewing the components.

FIG. 17 is a bottom lateral oblique perspective view of a table engagement system of the lateral decubitus precision patient support system.

FIG. 18 is a side lateral oblique perspective view of a treatment breast support of the lateral decubitus precision patient support system.

FIG. 19 is a top lateral oblique perspective view of a head support of the lateral decubitus precision patient support system.

FIG. 20 is a top lateral oblique perspective view of a head support of the lateral decubitus precision patient support system.

FIG. 21 is a top lateral oblique perspective view of a portion of a support body with patient support surface and an arm support of the lateral decubitus precision patient support system.

FIG. 22 is a top lateral oblique perspective view of a table engagement system of the lateral decubitus precision patient support system.

FIG. 23 is top lateral oblique perspective view of a lifting device of the lateral decubitus precision patient support system.

FIG. 24 is a side lateral oblique perspective view of an elevation module of the lateral decubitus precision patient support system.

FIG. 25 is a partially exploded, side lateral oblique perspective view of the lateral decubitus precision patient support system with a patient lying on the patient support surface.

FIG. 26 is a bottom lateral oblique perspective view of the lateral decubitus precision patient support system with a patient lying on the patient support surface.

### DETAILED DESCRIPTION OF THE INVENTION

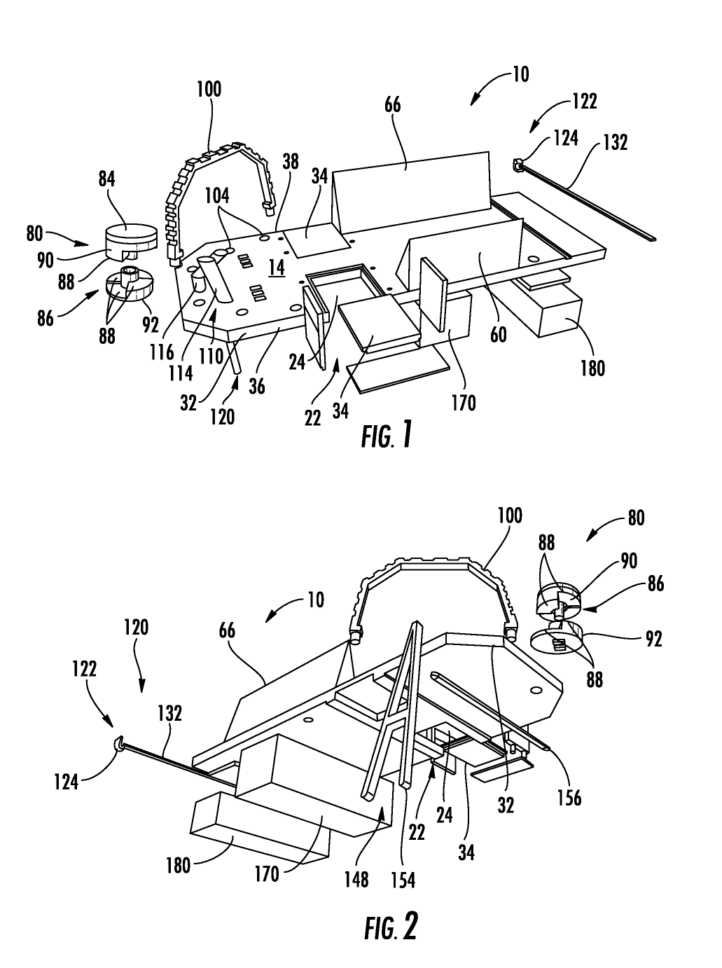

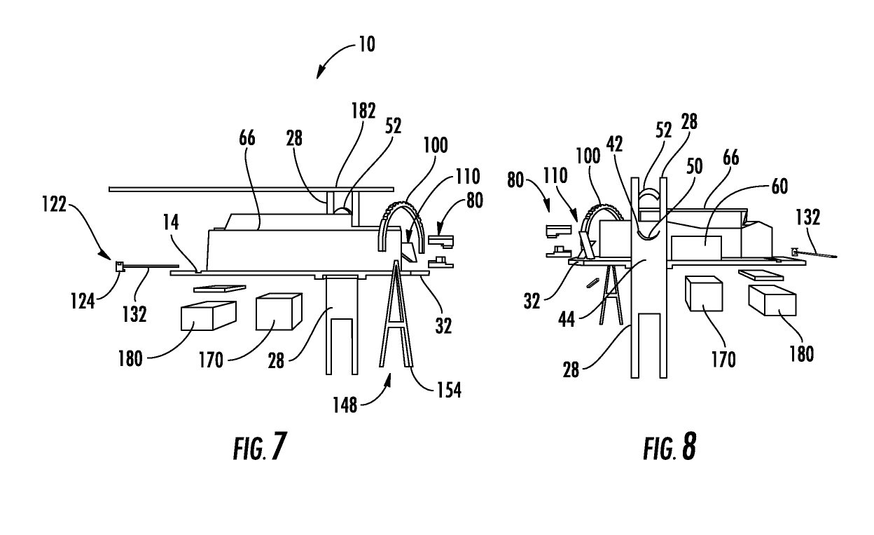

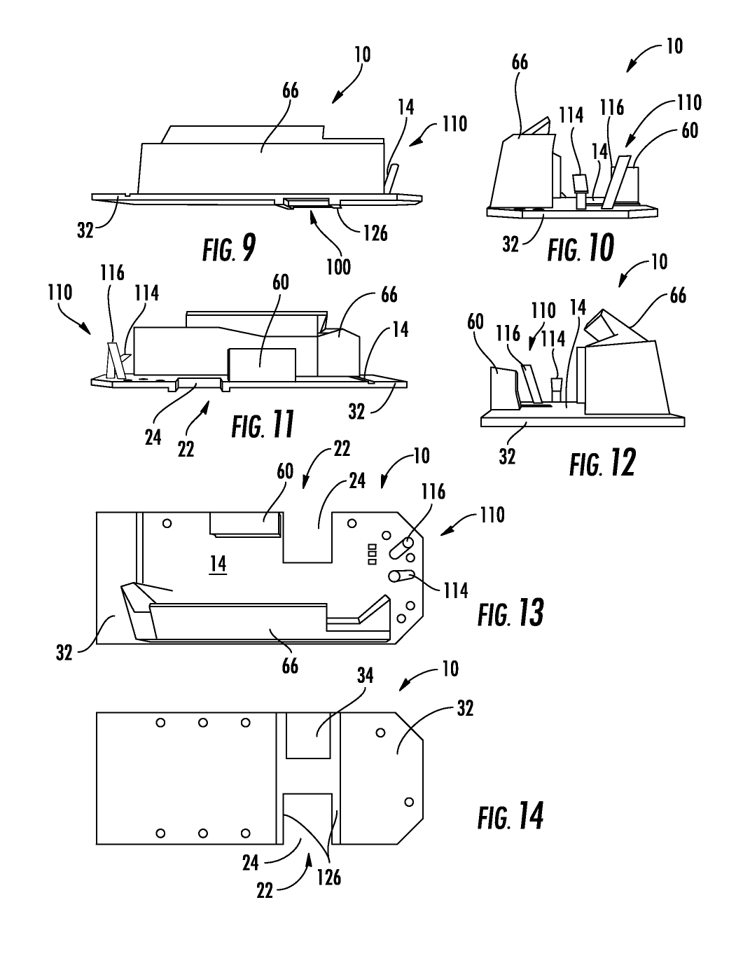

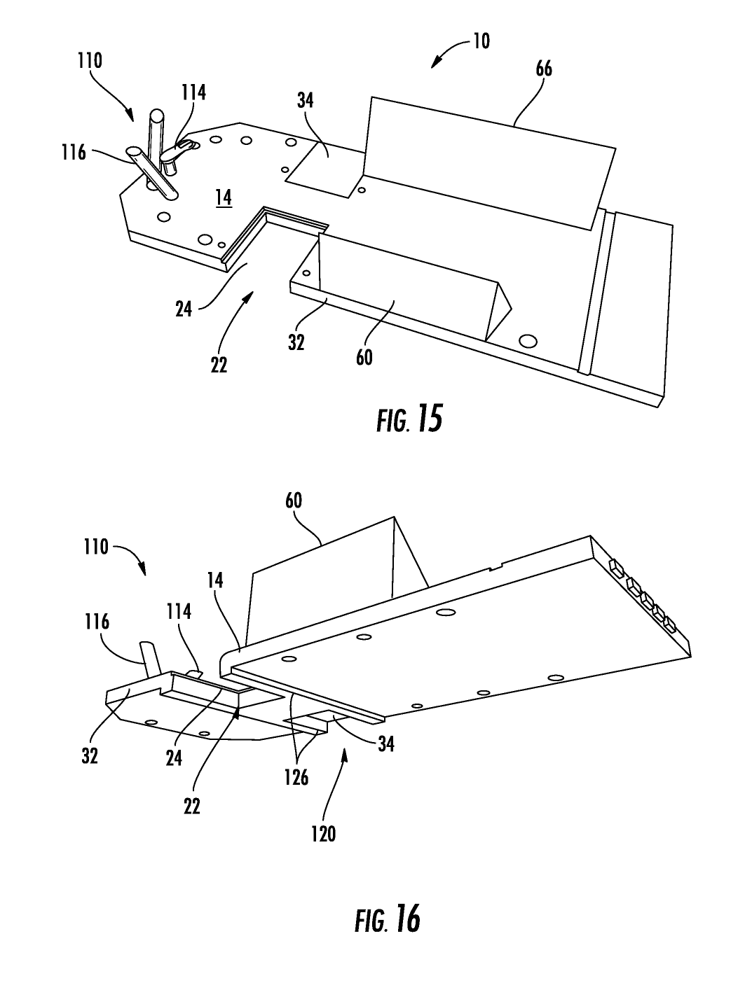

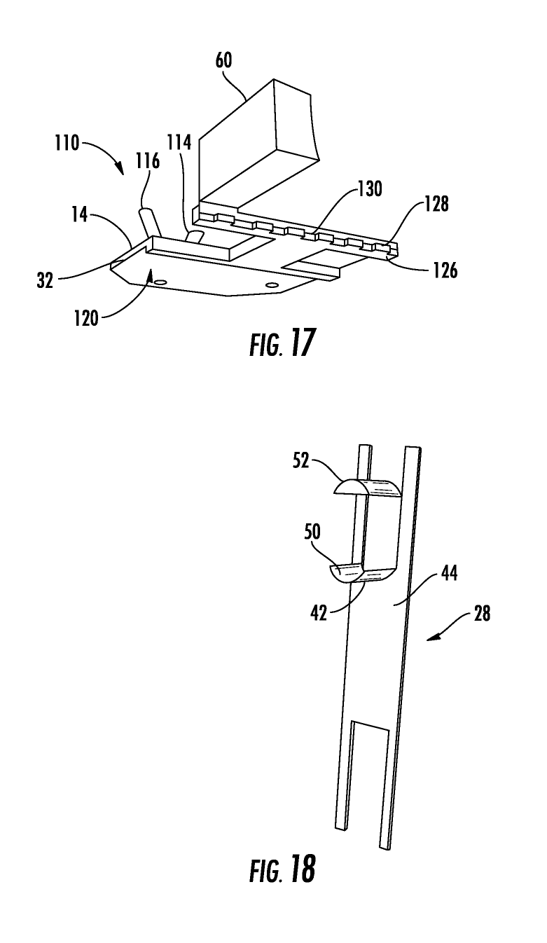

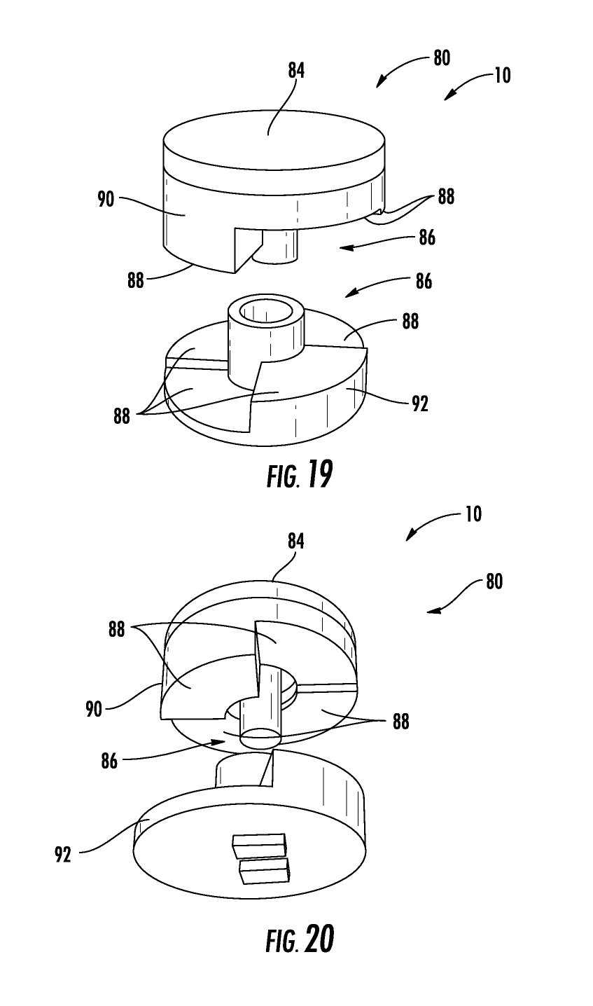

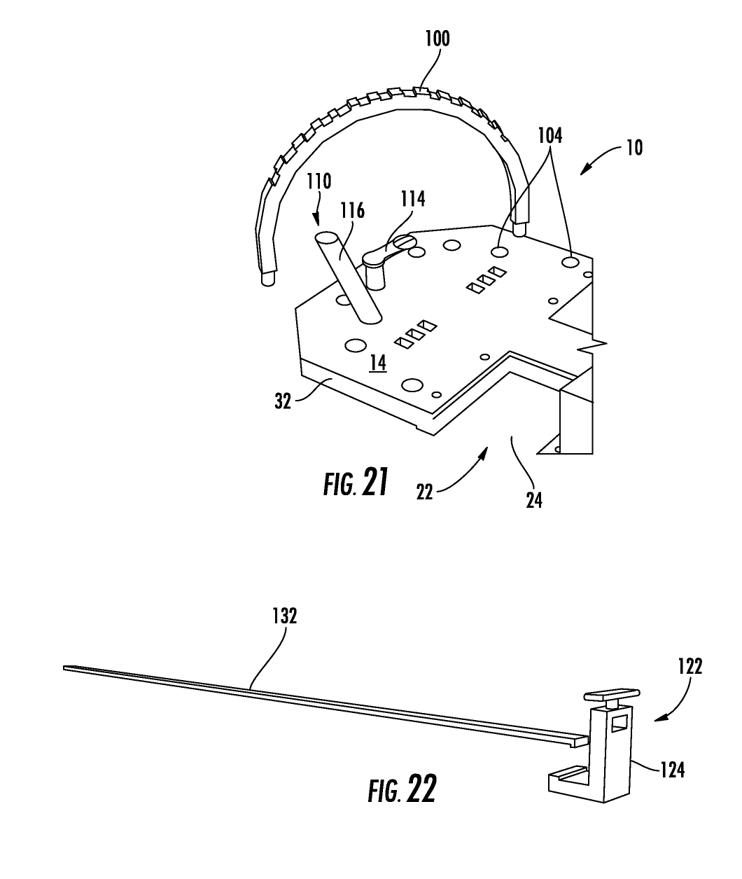

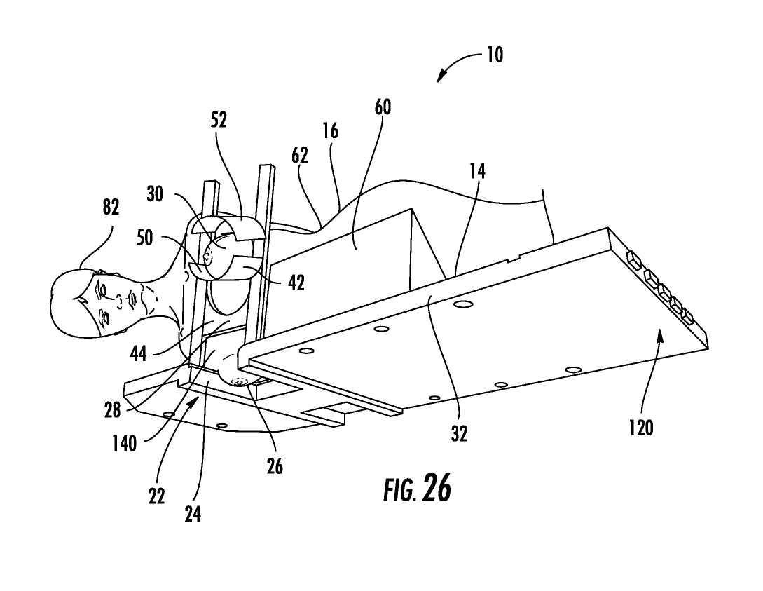

As shown in FIGS. 1-26, a lateral decubitus precision patient support system 10 and method for accurately and reproducibly positioning a patient's breast 12 relative to a patient support surface 14 to receive therapy, such as, but not limited to, radiation treatment, while the patient 16 is in a lateral decubitus position (lying on the patient's side) upon the patient support surface 14 is disclosed. The lateral decubitus precision patient support system 10 may be securely attached to a treatment table 20, as shown in FIG. 25, during use. The lateral decubitus precision patient support system 10 may include a contralateral breast module 22, as shown in FIG. 26, having at least one contralateral breast receiving area 24 into which a contralateral breast 26 of the patient 16 may reside during breast therapy. The lateral decubitus precision patient support system 10 may also include a treatment breast support 28, as shown in

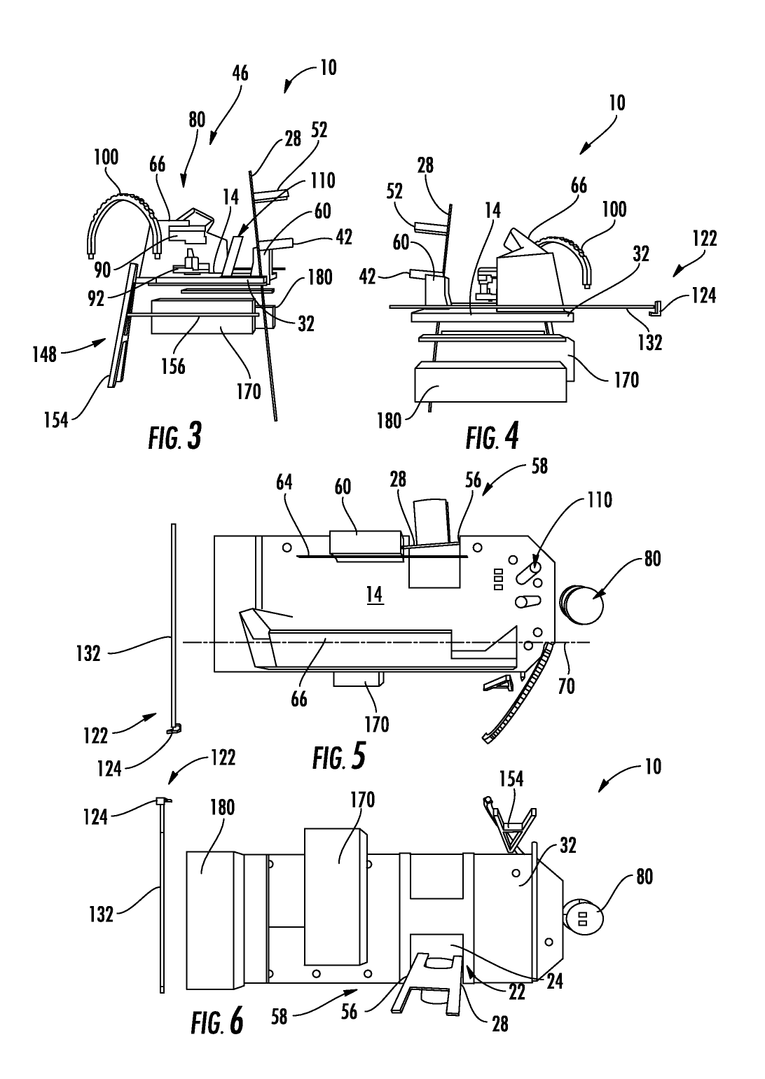

FIGS. 3-8 and 18, that contacts a treatment breast 30 of the patient 16 to separate the treatment breast 30 from the contralateral breast 26 of the patient 16 to limit exposure of the contralateral breast 26 to therapeutic procedures administered to the patient 16.

As shown in FIGS. 1-16 and 24-26, the lateral decubitus precision patient support system 10 for breast therapy may be formed from one or more support bodies 32 having at least one patient support surface 14 configured to support a patient in a lateral decubitus position on the patient support surface 14. The support body 32 may have any appropriate shape and configuration capable of partially or completely supporting a patient 16. The support body 32 may be formed from materials, such as, but not limited to, metals, plastics, carbon fiber and the like. The patient support surface 14 may be reside within a single plane or multiple planes. The patient support surface 14 may also have indentations to accommodate anatomical portions of a patient 16 when the patient is lying thereon. The patient support surface 14 may be formed from materials that provide comfort the patient, such as, but not limited to, deformable materials, such as memory foam and the like. The patient support surface 14 may be covered with one or more sterile coverings. In one embodiment, the support body and patient support surface 14 may be covered with a cushion 182, which may be, but is not limited to being, an adjustable cushion, or cradle.

The support body 32 and corresponding patient support surface 14 may be any one of numerous lengths. For instance, the support body 32 and the patient support surface 14 may be full length, which is slightly longer than a height of a patient, such as, but not limited to, about seven feet, a short length, which may be any length less than full length. In one embodiment, the short length of the support body 32 and the patient support surface 14 may be between 2.5 and 4.5 feet. In another embodiment, the support body 32 and the patient support surface 14 may have adjustable lengths. The support body 32 with the patient support surface 14 may have a generally flat patient support surface 14.

As shown in FIGS. 1, 2, 5, 6, 13-16 and 26, the lateral decubitus precision patient support system 10 may also include one or more contralateral breast modules 22 having at least one contralateral breast receiving area 24 into which a contralateral breast 26 of patient 16 resides during breast therapy. The contralateral breast receiving area 24 enables gravity to pull the contralateral breast 26 away from the treatment breast 30 to provide separation, thereby enabling the contralateral breast 26 from being exposed to therapy administered to the treatment breast 30. In at least one embodiment, the contralateral breast receiving area 24 of the contralateral breast module 22 may be positioned within the patient support surface 14. The contralateral breast receiving area 24 may have any appropriate shape and size. For instance, the contralateral breast receiving area 24 may be generally circular, rectangular, square or have another definable or abnormal shape. The size of the contralateral breast receiving area 24 may be adjustable to account for differences in sizes of breasts 12 between different patients.

The contralateral breast module 22 may include a contralateral breast support 34, as shown in FIGS. 1 and 15, configured to at least partially fill the contralateral breast receiving area 24 in the contralateral breast module 22 and support the contralateral breast 26 of the patient 16 before breast therapy. The contralateral breast support 34 may be removably attached to the contralateral breast module 22. In at least one embodiment, the contralateral breast support 34 may be removably attached to the patient support surface 14. The contralateral breast support 34 may be slidably attached to the patient support surface 14 such that the contralateral breast support 34 may be slidably removed to reveal the contralateral breast receiving area 24. The patient support surface 14 may include a contralateral breast support 34 on each of first and second edges 36, 38 of the patient support surface 14, whereby the first and second edges 36, 38 are positioned on opposite edges from each other. In such a position, the contralateral breast supports 34 on the first and second edges 36, 38 can accommodate the patient lying on the patient's right or left side.

As shown in FIGS. 3-8, 18, 25 and 26, the lateral decubitus precision patient support system 10 may also include a treatment breast support 28 that contacts a treatment breast of a patient 16 to separate the treatment breast 30 from the contralateral breast 26 of the patient 16 to limit exposure of the contralateral breast 26 to therapeutic procedures administered to the patient 16. The treatment breast support 28 may include one or more lower lifting supports 42 extending from a treatment breast support body 44 and configured to support a treatment breast 30 of the patient 16 at a higher position relative to the patient support surface 14 when the patient 16 is in the lateral decubitus position than the treatment breast 30 would naturally be positioned when the patient 16 is in the lateral decubitus position. A contact surface 50 of the lower lifting support 42 of the treatment breast support 28 that contacts the treatment breast 30 of the patient 16 may be formed from a deformable material to comfort the treatment breast 30 of the patient 16. The deformable material may be any material having the capacity to comfortably support the treatment breast 30. The deformable material may be, but is not limited to, memory foam. At least a portion of the lower lifting support 42 that contacts the treatment breast 30 of the patient 16 may be flexible to readily conform to the shape of the treatment breast 30 to comfort the treatment breast 30 of the patient. The flexible material may be a mesh, such as, but not limited to, a plastic mesh, a fabric and the like. The lower lifting support 42 for supporting the treatment breast 30 can be tightly clipped onto the top of the treatment breast support 28 which then may be mounted to the support body 32 through couplers. During use, the treatment breast 30 is positioned at an elevated, horizontal, forward-extended, and reproducible position with a reduced breast thickness and less skin fold. The lower lifting support 42 of the treatment breast support 28 may be positioned at one of a number of different heights for a good fit to the patient 16. In at least one embodiment, the lower lifting support 42 may be made with a thin fine and rigid net material with holes, which may be, but are not limited to being, sized to be no more than five mm each. The lower lifting support 42 may be indexed relative to the treatment breast support 28 so that a position can be precisely determined, recorded and replicated.

The treatment breast support 28 may also include one or more upper lifting supports 52 to hold a treatment breast 30 in a forward extended, reproducible position for therapy, such as, but not limited to, multi-day radiation therapy treatments. The treatment breast support 28 may be adjustable relative to the lower lifting support 42. The upper lifting support 52 and the lower lifting support 42 may be moved closer together to contact a treatment breast 30 positioned between the lower and upper lifting supports 42, 52. The lower and upper lifting supports 42, 52 may enable the treatment breast 30 to be accurately positioned at a horizontal and forward extending position to receive treatment, such as, but not limited to, radiation treatment or diagnostic imaging. The lower lifting support 42 may extend from the treatment breast support body 44 laterally away from the treatment breast support body 44 and away from a patient receiving area 46 on the patient support surface 14.

In at least one embodiment, the treatment breast support 28 may be removably attachable to the support body 32 of the lateral decubitus precision patient support system 10. The treatment breast support body 44 may be coupled to the support body 32 via a connection system 58. The connection system 58 may be any appropriate connection system configured to attached the treatment breast support body 44 to the support body 32 having the patient support surface 14. The connection system 58 may be a releaseably attachable system. In at least one embodiment, the connection system 58 may be formed from a receiving slot 56 positioned within the patient support surface 14 of the support body 32. The slot 56 may receive an end of the treatment breast support body 44 and hold the treatment breast support body 44 in a position extending away from the patient support surface 14. The treatment breast support body 44 may be oriented to be generally orthogonal to the patient support surface 14.

The lateral decubitus precision patient support system 10 may also include one or more contralateral breast holder devices 140, as shown in FIG. 26, configured to bind the contralateral breast 26 of the patient 16 to the patient's body while pushing the contralateral breast 26 away from treatment fields directed at the treatment breast 30 of the patient 16. The contralateral breast holder device 140 may be a portion of the treatment breast support 28. The contralateral breast holder device 140 may be permanently or releaseably coupled to the treatment breast support 28, or alternatively, may be integrally formed with the treatment breast support 28. The contralateral breast holder device 140 may be formed from a deformable material, such as, but not limited to a deformable plastic material. The contralateral breast holder device 140 may be rigidly attached to the treatment breast support 28 such that the contralateral breast holder device 140 is limited in movement and presses against a contralateral breast 26. Alternatively, the contralateral breast holder device 140 may be hingedly attached to the treatment breast support 28 such that the contralateral breast holder device 140 is biased, such as spring biased, towards the contralateral breast 26.

As shown in FIGS. 1-5, 8, 10-13, 15-17, 25, and 26, the lateral decubitus precision patient support system 10 may also include an adjustable abdominal support 60 for maintaining position of a torso 62 of the patient 16 relative to the patient support surface 14, and thereby maintain position of a treatment breast 30 in a repeatable position and to prevent torso 62 rotation. The abdominal support 60 may have any appropriate size and shape to assist in positioning the patient 16 in a repeatable position relative to the patient support surface 14 and the treatment breast support 28. The abdominal support 60 may be configured to resemble the abdominal region of a human torso 62. The abdominal support 60 may be curved about a longitudinal axis 64 of the support 60. In at least one embodiment, the abdominal support 60 may have a generally rectangular cross-section and may be shaped as a block. The abdominal support 60 may be removably attachable on the patient support surface 14 of the support body 32.

As shown in FIGS. 1, 3-5, 7-13 and 15, the lateral decubitus precision patient support system 10 may also include an adjustable back support 66 for maintaining position of a torso 62 of the patient 16 relative to the patient support surface 14, and thereby maintain position of a treatment breast 30 in a repeatable position and to prevent torso 62 rotation. The back support 66 may have any appropriate size and shape to assist in positioning the patient 16 in a repeatable position relative to the patient support surface 14 and the treatment breast support 28. The back support 66 may be configured to resemble the back region of a human torso 62. The back support 66 may be curved about a longitudinal axis 70 of the back support 66. In at least one embodiment, the back support 66 may have a generally rectangular cross-section and may be shaped as an elongated board. The back support 66 may be removably attachable on the patient support surface 14 of the support body 32.

As shown in FIGS. 1, 2, 5-8, 19, 20 and 25, the lateral decubitus precision patient support system 10 may also include a head support 80 for maintaining position of a head 82 of the patient 16 relative to the patient support surface 14, and thereby maintain position of a treatment breast 30 in a repeatable position. The head support 80 may have any appropriate size and shape to assist in positioning the patient 16 in a repeatable position relative to the patient support surface 14 and the treatment breast support 28. The head support 80 may be configured to resemble the head 82 of the patient 16. The head support 80 may include an indentation to receive a lateral side of the patient's head 82. In at least one embodiment, the head support 80 may have a generally circular, linear support surface 84. The head support 80 is not limited to this shape or configuration but may have other shapes as well. The head support 80 may be removably attachable on the patient support surface 14 of the support body 32.

The head support 80 may be held in position relative to the patient support surface 14 with a support system 86. The head support 80 may maintain position of the head 82 of the patient 16 relative to the patient support surface 14, and thereby maintain position of a treatment breast 30 in a repeatable position. The support system 86 makes the head support 80 adjustable such that the head contact surface 84 is adjustable vertically upward relative to the patient support surface 14. The support system 86 may be any device capable of adjusting the height of the head support surface 84 relative to the patient support surface 14. The support system 86 may be adjustable vertically upward or downward relative to the patient support surface 14 via partial circumferential steps 88 on top and bottom head support members 90, 92 forming the head support 80. The circumferential steps on the top and bottom head support members 90, 92 each have a different longitudinal orientation relative to adjacent steps 88. The height of the head support surface 84 may be adjusted by rotating the top head support member 90 relative to the bottom head support member 92. On outer surface of the head support 80 may be a deformable surface for comforting the patient's head 82. In one embodiment, the top head support member 90 may have four equal incremented steps 88, and the bottom head support member 92 may have four equal incremental steps 88. In other embodiments, the number of steps 88 may vary and may vary between the top and bottom head support members 90, 92.

The engaging surfaces of both the top and bottom head support members 90, 92, where the lower face of the top piece has four equal incremented steps except between the last and the first step, a large hollow cylinder indent for receiving and a small cylindrical post extended downward for inserting into the base piece of the holder. The bottom head support member 92 may have two rectangular posts attached to the bottom of the base piece for support body 32 attachment. The steps 88 on both the top and bottom head support members 90, 92 may be mirror images of each other and may be configured to nest with each other. The steps 88 may be used to change the height of the head support 80 using different steps 88 at different rotations of the top head support member 90 relative to the bottom head support member 92.

As shown in FIGS. 1-5, 7, 8 and 21, the lateral decubitus precision patient support system 10 may also include an adjustable arm support 100 for supporting a lesion side arm 102 of the patient 16. The adjustable arm support 100 may be rotatable about a pivot point 104 on the patient support surface 14. The adjustable arm support 100 may have any appropriate size and shape to assist in positioning the patient 16 in a repeatable position relative to the patient support surface 14 and the treatment breast support 28. In at least one embodiment, the adjustable arm support 100 may be generally curved, such as, but not limited to, a semicircular shape to support the lesion side arm 102. The adjustable arm support 100 may be rotated to be within close proximity to the patient 16.

As shown in FIGS. 1, 3-5, 7, 8, 9-13, 15, 16, 17, 21 and 25, the lateral decubitus precision patient support system 10 may also include one or more adjustable handles 110 for gripping by a patient's hands 112 to provide stability and comfort to the patient 16 and to create a reproducible position of the patient's treatment breast 30. The system 10 may include a right hand handle 114 and a left hand handle 116. The right and left hand handles 114, 116 may be positioned at an end of the patient support surface 14 to which the head support 80 is attachable. The right and left hand handles 114, 116 may extend orthogonally from the patient support surface 14 or may be positioned nonorthogonally and nonparallel relative to the patient support surface 14. The right and left hand handles 114, 116 may be lockable in place and may include an indexing system such that the position of the right and left hand handles 114, 116 may be recorded and replicated for each therapy session for the patient 16. The indexing system may be a series of marks on the patient support surface 14 at the right and left hand handles 114, 116 and corresponding marks on the right and left hand handles 114, 116 themselves. The marks may be identified thereon to facilitate recording the position and replicating the position during the next therapy treatments.

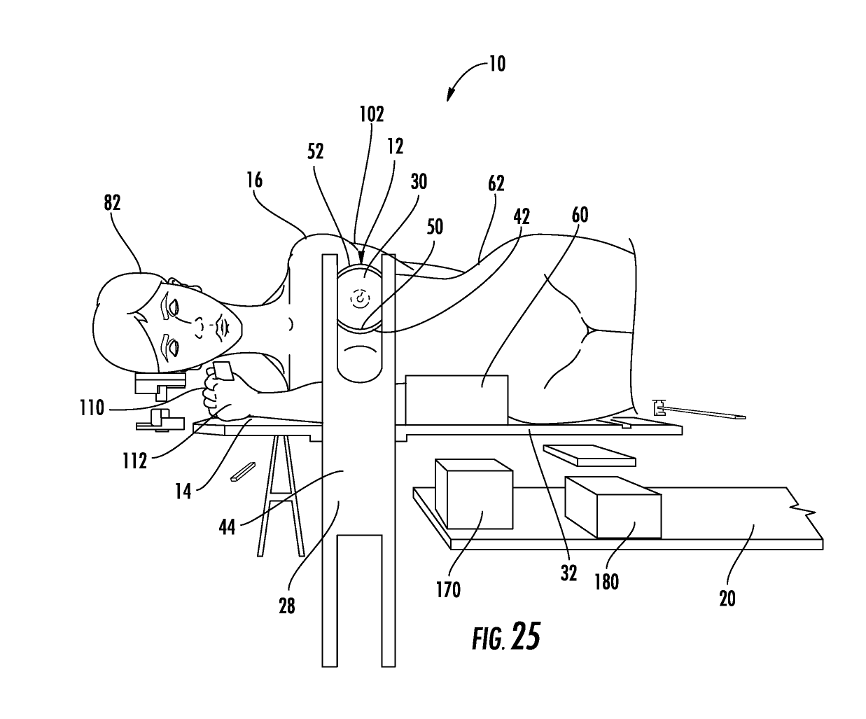

As shown in FIGS. 24 and 25, the lateral decubitus precision patient support system 10 may also include one or more shoulder pads 170 for maintaining position of a head 82 and torso 62 of the patient 16 relative to the patient support surface 14, and thereby maintain position of a treatment breast 30 in a repeatable position. The shoulder pad 170 may have any appropriate size and shape to assist in positioning the patient 16 in a repeatable position relative to the patient support surface 14 and the treatment breast support 28. The shoulder pad 170 may be configured to resemble the shoulder and upper back region of a human torso 62. In at least one embodiment, the back support 66 may have a generally rectangular cross-section and may be a generally elongated block. The shoulder pad 170 may be removably attachable on the patient support surface 14 of the support body 32. The shoulder pad 170 may have an inclined outer surface 174 configured to mate with an outer shoulder pad 172. The outer shoulder pad 172 may have an inner surface 176 that is inclined and may be a mirror image of the outer surface 174. As such, the outer shoulder pad 172 may be moved laterally relative to the shoulder pad 170, causing an outer surface 178 of the outer shoulder pad 172 to move vertically upward or downward relative to the patient support surface 14 without the orientation of the outer surface 178 of the outer shoulder pad 172 changing relative to the patient support surface 14.

As shown in FIGS. 24 and 25, the lateral decubitus precision patient support system 10 may also include one or more leg pads 180 for maintaining position of the torso 62 of the patient 16 relative to the patient support surface 14, and thereby maintain position of a treatment breast 30 in a repeatable position. The leg pad 180 may have any appropriate size and shape to assist in positioning the patient 16 in a repeatable position relative to the patient support surface 14 and the treatment breast support 28. The leg pad 180 may be configured to resemble the legs of a patient 16. In at least one embodiment, the leg pad 180 may have a generally rectangular cross-section and may be a generally elongated block. The leg pad 180 may be removably attachable on the patient support surface 14 of the support body 32.

As shown in FIGS. 22-25, the lateral decubitus precision patient support system 10 may also include one or more table engagement systems 120 configured to couple the support body 32 and patient support surface 14 to a table, such as, but not limited to, an examination table, a therapy table, or other table. The support body 32 may be coupled to already existing tables so that a new table need not be purchased, and the table may be used for other purposes other than supporting the support body 32 as well. The table engagement system 120 may have at least one connector 122 for attaching the patient support surface 14 to an existing table. The connector 122, may be, but is not limited to being, a releasable clamp 124.

The table engagement system 120 may also include one or more enhancement members 126 for supporting the support body 32 and for enhancing the ability of the support body 32 to be coupled to a table. The enhancement member 126 may include one or more keys 128 configured to be received in one or more keyways 130 on a table, or vice versa. In at least one embodiment, the enhancement member 126 may be positioned proximate to the contralateral breast receiving area 24 found in the patient support surface 14 of the support body 32. The enhancement member 126 may be generally a planar member coupled to a bottom surface of the support body 32. In other embodiments, the enhancement member 126 may be coupled, permanently or releaseably, to other aspects of the lateral decubitus precision patient support system 10.

In at least one embodiment, the table engagement system 120 may also include a cross bar 132 and one or more connectors 122 for attaching the table engagement system 120 to the support body 32. The table engagement system 120 may be tailored for the receiving end of a table, such as, but not limited to, an existing table. For a short support body 32, the inferior border of the support body 32 may terminate at the enhancement member 127 to reduce its pitch force to the table base. The support body 32 may be constructed of sufficiently rigid and strong material such as, but not limited to, a high-strength carbon fiber or lightweight plastic, with or without metal bar enhancement in its inferior portion, and the like. The material may have a width between about 50-64 cm and a length between about 100-130 cm for the short mode and between about 140 -160 cm for the long mode. The cross bar 132 may be placed in the grove on the surface at inferior portion of the main support body 32, may extend laterally beyond the borders of the support body 32 and the connectors 122 and may rest in the holes of the connectors 122. By tightening top screws of connectors 122, the cross bar 132 may be compressed against the surface of the support body 32, thereby firmly attaching support body 32 to the table underneath. The downward raised ends of the cross bar 132 may further prevent the cross bar 132 from sliding off the connectors 122.

The lateral decubitus precision patient support system 10 may also include one or more contralateral breast holder devices 140, as shown in FIG. 26, configured to bind the contralateral breast 26 of the patient 16 to the patient's body while pushing the contralateral breast 26 away from treatment fields directed at the treatment breast 30 of the patient 16. The contralateral breast holder device 140 may be a portion of the treatment breast support 28. The contralateral breast holder device 140 may be permanently or releaseably coupled to the treatment breast support 28, or alternatively, may be integrally formed with the treatment breast support 28.

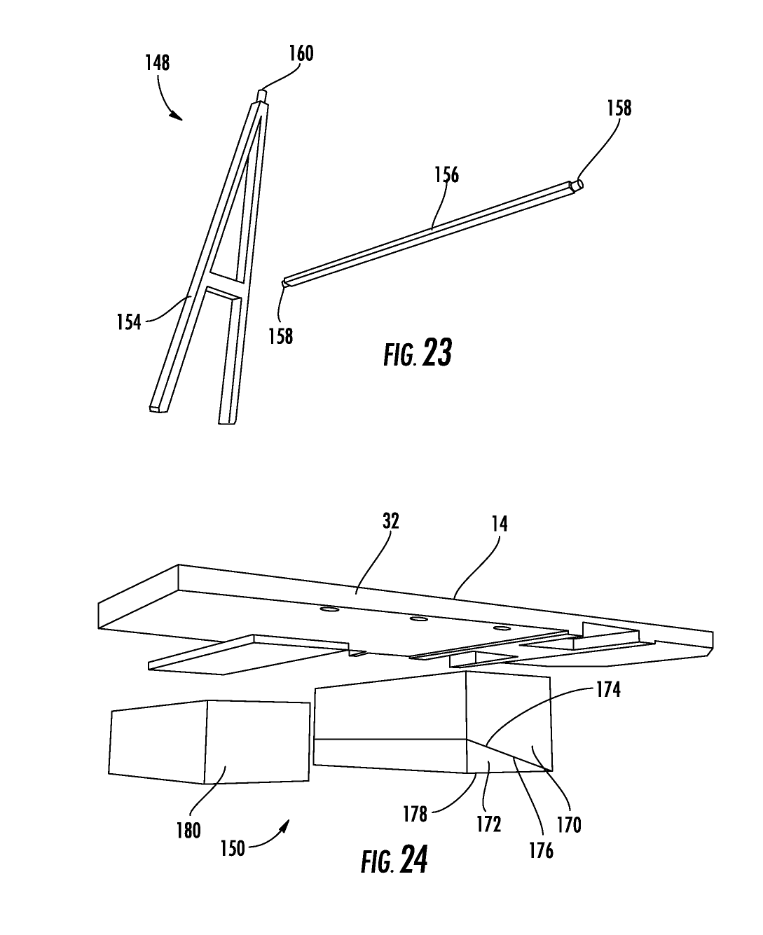

The lateral decubitus precision patient support system 10 may also include one or more elevation modules 148, as shown in FIGS. 23 and 24, for elevating the support body 32 above a surface upon which the support body 32 rests to create a gap there between to insert one or more elevation pads 150 to change the elevation of the patient 16. The lateral decubitus precision patient support system 10 may also include one or more lifting devices 152 that is configured to suspend one end of the support body 32 and the patient 16 above a surface upon which the support body 32 rests to create a gap there between to insert one or more elevation pads 150. In at least one embodiment, the lifting device 150 may be formed from a frame 154, which may be configured as an A frame or other appropriate configuration. The lifting device 150 may also include a lifting bar 156 that is usable in conjunction with the frame 154 to lift an end of the support body 32. The lifting bar 156 may include one or more coupling orifices 158 configured to mate with a coupling post 160 on the frame 154.

The lifting device 152 may include a lifting bar 156 and two holding frames 154 (only one frame 154 is shown). The lifting bars 156 may be placed under the inferior or superior portion of the support body 32, cross the lateral borders of the support body 32, and then may be mounted to rest on the top of each side frame around the top coupling posts 160. The top coupling posts 160 of the two holding frames in the holding position may be tilted by about 15 degrees toward the center of the table to form a stable lifting platform. The lifting device 152 may be constructed with strong, rigid, and light material, such as, but not limited to, a high-strength aluminum alloy with slippery-proof foot pads. During use, one end of the baseboard and the patient 16 may be lifted at a relatively higher position to create a space for the elevation pads 150 to be inserted under the support body 32, resulting in increased gap between the support body 12 and the existing table.

The elevation module 148 may include different shaped and sized columns and pads which may be used to tailor the fit of the lateral decubitus precision patient support system 10 to the patients 16. The elevation pads 150 may be, but are not limited to being, made of light and relatively rigid material, each with strength to support 150 kg weight for no less than 60 minutes without significant distortion. When in use, the elevation pads 150 can be securely stacked on each other to elevate the lateral decubitus precision patient support system 10 and the patient table, hence creating a gap from the existing table for the contralateral breast 26 to hang. A typical configuration of elevation pads 150 may include a stack of two superior pads having dimensions such as, but not limited to, 20 cm×20 cm×50 cm full-size pad with a wedged pad separated from its inferior lower corner and two stacked inferior pads, such as a 20 cm×18 cm×50 cm thick pad and a 20 cm×2 cm×50 cm thin pad.

All components of the lateral decubitus precision patient support system 10 may be formed from materials, such as, but not limited to, metals, such as, but not limited to, stainless steel, titanium, aluminum and the like, plastics, and carbon fiber matrices. The materials may have sufficient strength and rigidity to support the patient and to form a robust configuration. The materials may also be lightweight. In one embodiment, all components are formed from the same material. In other embodiments, one or more, or all of the components may be formed from different materials relative to each other.

During use, the lateral decubitus precision patient support system 10 may be used to position a patient 16 in a repeatable position such that therapies, such as, but not limited to radiation therapy, may be administered to the treatment breast 30 of a patient 16. The success of such treatments is due, in part, to the ability to replicate treatments to the target tissue in the treatment breast 30 and to provide uniform exposure to the target tissue. Being able to adjust the components of the lateral decubitus precision patient support system 10, such as, but not limited to, the treatment breast support 28, the contralateral breast support 34, the adjustable abdominal support 60, adjustable back support 66, head support 80, adjustable arm support 100, adjustable handles 110, elevation pad 150, shoulder pad 170 and leg pad 180, enables the position of a patent 16 within the system 10 to be replicated with a high degree of accuracy during each therapy treatment session. One or more, or all of these components of the lateral decubitus precision patient support system 10 may be indexed so that the precise position of each component can be determined relative to an indexing reference, and such information may be recorded and used in future therapy sessions to setup the lateral decubitus precision patient support system 10 for that particular patient 16. As such, a high degree of precision of patient orientation and position relative to the support body 32 and the patient support surface 14 may be obtained.

In accordance with another aspect of the lateral decubitus precision patient support system 10, a method for comfortably and reproducibly positioning a patient's breast 12 to receive radiation on a modern treatment table is disclosed. The method may include the attaching the patient support surface 14 of the lateral decubitus precision patient support system 10 to a table by (a) placing a long support body 32 on the top surface of the table with its bottom enhancement members 126 extended just beyond the superior border of the table, and securing support body 32 against the table using the table engagement system 120, which may be formed from a compressing cross bar 132 and two table attachment connectors 122, or by (b) directly attaching a support body 32 to the superior end of the table base using the table engagement system 120. The method may also include directing the patient to sit on the left side of lateral decubitus precision patient support system 10 in the lower section and to lay down on the right side upon the lateral decubitus precision patient support system 10. The method may include adjusting the patient's head 82 on the head support 80. The method may also include positioning the patient's hands 112 on the adjustable handles 110 and arms on the adjustable arm support 100. The method may include removing the contralateral breast support 34 while gently hanging and pulling the left breast 12 downward. The method may also include placing the right breast 12 onto the lower lifting support 42 at an appropriate height, and securing the treatment breast support body 44 to the support body 32 and firmly contacting the lower and upper lifting supports 42, 52 to the treatment breast 30 of the patient 16 the treatment breast 30 is positioned at an elevated, horizontal and forward extended position and while pushing the contralateral breast 26, which is the lower breast, downward and away from potential exposure to treatment fields. The method may include placing and adjusting the abdominal and back support 60, 66 on the support body 32 to prevent the torso 62 from rotation. The method may also include forming the patient body contours for the adjustable cushion 182 between the patient 16 and the support body 32. The method may include positioning radiation treatment center relative to the treatment breast 30. The method may also include verifying and adjusting the treatment breast 30 position relative to the planned treatment fields by in-room imaging. The method may include irradiating the treatment breast 30. The method may also include marking and indexing the patient's position if needed. Identical method may be derived and applied to the right breast setup.

In accordance with another aspect of the lateral decubitus precision patient support system 10, a method for comfortably and reproducibly positioning a patient's breast 12 to receive radiation is disclosed. The method may take place with the lateral decubitus precision patient support system 10 used together with any table, such as, but not limited to, an imaging or a treatment table which limits the support body 32 of the lateral decubitus precision patient support system 10 from being extended superiorly beyond the table. The method may include placing the support body 32 of the lateral decubitus precision patient support system 10 to the center of the table and accordingly inserting a full-size shoulder pad 170 and a thin leg pad 180 under the support body 32, so that the support body 32 is moderately tilted up at its superior end. The method may also include directing the patient 16 to sit on the left side of the lateral decubitus precision patient support system 10 in the lower section and to lie down on the right side upon the lateral decubitus precision patient support system 10. The method may include inserting a lifting bar 156 under the inferior portion of the support body 32 against the superior border of the thin elevation pad 150, and placing the holding frames 154 parallel on each side with top tilted towards the center by about 15 degrees. The method may also include raising the table to allow the lifting bar 156 to be mounted and rest on the top of side frames 154 around the top posts 160. The method may include lowering the table while the lateral decubitus precision patient support system 10 and the patient 16 are held at a desired height by the lifting bar 156 and frames 154, and after inserting the thick leg pad 180 and the wedged shoulder pad 172, raising the table to dismount the lifting bar 156 and frames 154. The method may also include adjusting the patient's head 82 on the head support 80. The method may include positioning the patient's hands 112 and arms in contact with the adjustable handles 110 and the adjustable arm support 100. The method may also include removing the contralateral breast support 34 while gently pulling the contralateral breast 26 downward. The method may include securing the treatment breast 30 on the lower lifting support 42 of the treatment breast support body 44 at the appropriate height and securing the treatment breast support body 44 to the support body 32 and firmly against the contour of the patient's torso 62 while pushing the contralateral breast 26, which is the lower breast, downward and away from potential exposure to treatment fields. The method may also include placing and adjusting the abdominal and back supports 60, 66 on the support body 32 to prevent the patient's torso 62 from rotating. The method may also include forming the patient body contours for the adjustable cushion 182 between the patient 16 and the support body 32. The method may include positioning radiation treatment equipment or simulation region relative to the treatment breast 30. The method may also include performing the treatment verifications or simulation scans and marking and indexing the patient's position if needed. This process may be applied to a patient's other breast as well.

The lateral decubitus precision patient support system 10 may be used with a big bore CT imaging system. The lateral decubitus precision patient support system 10 may also be constructed so that it will not interfere with the big bore CT imaging system or other imaging modalities, including: a cone beam CT, MRI and PET. The support body 32 can also be manufactured to fit variety of table configurations, including a full-length or long and a half-length or short tables, tables with and without side rails, as long as the combined downward pitching force and total weight does not exceed the table's capacity. The lateral decubitus precision patient support system 10 may also be used with a conformable patient's immobilization cradle or cushion, respiratory control techniques, patient's surface, structure monitoring systems or other devices available in radiation therapy to enhance its immobilization process for the patient 16 receiving the treatment. The lateral decubitus precision patient support system 10 may be further combined with a respiration gating technique, a respiration holding or a controlling technique, a multidimensional positioning technique, in-room imaging techniques, surface monitoring techniques, and other advanced position control and monitoring techniques to improve positioning precision and reproducibility during entire treatment courses. The lateral decubitus precision patient support system 10 may further promote the separation of the contralateral breast 26 from the treatment breast 30 and radiation field and may possibly decrease the risk of secondary cancer in the healthy breast. The lateral decubitus precision patient support system 10 may also expand the lung volume through the downward shift of the mediastinum including the heart towards the non-treatment side by gravity, possibly resulting in lower lung and heat toxicity. The lateral decubitus precision patient support system 10 may also provide a patient 16 with more freedom through additional possible arm positions. In addition to the common position of holding the lesion side arm 102 above the patient's head 82 to avoid being irradiated, the lateral decubitus precision patient support system 10 may enable the patient's arm 102 to be placed behind the patient's back without being irradiated, which may create more stable and reproducible setups, especially for the patients 16 with limited or difficult arm motion.

The foregoing is provided for purposes of illustrating, explaining, and describing embodiments of this invention. Modifications and adaptations to these embodiments will be apparent to those skilled in the art and may be made without departing from the scope or spirit of this invention.

I claim:

1. A lateral decubitus precision patient support system for breast therapy, comprising:
at least one patient support surface configured to support a patient in a lateral decubitus position on the patient support surface;
at least one contralateral breast module having at least one contralateral breast receiving area into which a contralateral breast of patient resides during breast therapy.

2. The lateral decubitus precision patient support system of claim 1, wherein the at least one contralateral breast receiving area of the at least one contralateral breast module is positioned within the at least one patient support surface.

3. The lateral decubitus precision patient support system of claim 2, wherein the at least one contralateral breast module further comprises a removable contralateral breast support configured to at least partially fill the contralateral breast receiving area in the at least one contralateral breast module and support the contralateral breast of patient before breast therapy.

4. The lateral decubitus precision patient support system of claim 1, further comprising a treatment breast support that contacts a treatment breast of a patient to separate the treatment breast from the contralateral breast of patient to limit exposure of the contralateral breast to therapeutic procedures administered to the patient.

5. The lateral decubitus precision patient support system of claim 4, wherein the treatment breast support comprises at least one lower lifting support extending from a treatment breast support body and configured to support a treatment breast of patient at a higher position relative to the at least one patient support surface when the patient is in the lateral decubitus position than the treatment breast would naturally be positioned when the patient is in the lateral decubitus position.

6. The lateral decubitus precision patient support system of claim 5, wherein the treatment breast support further comprises at least one upper lifting support that is adjustable relative to the lower lifting support such that a treatment breast is held in a forward extended, reproducible position for therapy.

7. The lateral decubitus precision patient support system of claim 5, wherein a contact surface of the at least one lower lifting support of the treatment breast support that contacts the treatment breast of the patient is formed from a deformable material to comfort the treatment breast of the patient.

8. The lateral decubitus precision patient support system of claim 5, wherein at least a portion of the at least one lower lifting support that contacts the treatment breast of the patient is flexible to readily conform to the shape of the treatment breast to comfort the treatment breast of the patient.

9. The lateral decubitus precision patient support system of claim 4, wherein the least one lower lifting support extends from the treatment breast support body laterally away from the treatment breast support body and away from a patient receiving area on the patient support surface.

10. The lateral decubitus precision patient support system of claim 1, further comprising an adjustable abdominal support for maintaining position of a torso of the patient relative to the at least one patient support surface, and thereby maintain position of a treatment breast, in a repeatable position.

11. The lateral decubitus precision patient support system of claim 1, further comprising an adjustable back support for maintaining position of a torso of the patient relative to the at least one patient support surface, and thereby maintain position of a treatment breast, in a repeatable position.

12. The lateral decubitus precision patient support system of claim 1, further comprising a head support for maintaining position of a head of the patient relative to the at least one patient support surface, and thereby maintain position of a treatment breast, in a repeatable position.

13. The lateral decubitus precision patient support system of claim 1, wherein the head support is adjustable such that the head contact surface is adjustable vertically upward relative to the at least one patient support surface.

14. The lateral decubitus precision patient support system of claim 1, further comprising an adjustable arm support for supporting a lesion side arm of the patient, wherein the adjustable arm support is rotatable about a pivot point on the at least one patient support surface.

15. The lateral decubitus precision patient support system of claim 1, further comprising adjustable handles for gripping by a patient's hands to provide stability and comfort to the patient and to create a reproducible position of a treatment breast.

16. The lateral decubitus precision patient support system of claim 1, further comprising a table engagement system having at least one connector for attaching the at least one patient support surface to a table.

17. The lateral decubitus precision patient support system of claim 1, further comprising a breast holder device configured to bind the contralateral breast of the patient to the patient's body while pushing the contralateral breast away from treatment fields directed at a treatment breast of the patient.

18. A lateral decubitus precision patient support system for breast therapy, comprising:
at least one patient support surface configured to support a patient in a lateral decubitus position on the patient support surface;
at least one contralateral breast module having at least one contralateral breast receiving area into which a contralateral breast of patient resides during breast therapy;
a treatment breast support that contacts a treatment breast of a patient to separate the treatment breast from the contralateral breast of patient to limit exposure of the contralateral breast to therapeutic procedures administered to the patient;
wherein the treatment breast support comprises at least one lower lifting support extending from a treatment breast support body and configured to support a treatment breast of patient at a higher position relative to the at least one patient support surface when the patient is in the lateral decubitus position than the treatment breast would naturally be positioned when the patient is in the lateral decubitus position; and
a breast holder device configured to bind the contralateral breast of the patient to the patient's body while pushing the contralateral breast away from treatment fields directed at a treatment breast of the patient.

19. The lateral decubitus precision patient support system of claim 18, wherein the least one lower lifting support extends from the treatment breast support body laterally away from the treatment breast support body and away from a patient receiving area on the patient support surface.

20. A lateral decubitus precision patient support system for breast therapy, comprising:
at least one patient support surface configured to support a patient in a lateral decubitus position on the patient support surface;
at least one contralateral breast module having at least one contralateral breast receiving area into which a contralateral breast of patient resides during breast therapy;
a treatment breast support that contacts a treatment breast of a patient to separate the treatment breast from the contralateral breast of patient to limit exposure of the contralateral breast to therapeutic procedures administered to the patient;
wherein the treatment breast support comprises at least one lower lifting support extending from a treatment breast support body and configured to support a treatment breast of patient at a higher position relative to the at least one patient support surface when the patient is in the lateral decubitus position than the treatment breast would naturally be positioned when the patient is in the lateral decubitus position, and at least one upper lifting support that is adjustable relative to the at least one lower lifting support such that a treatment breast is held in a forward extended, reproducible position for therapy;
a breast holder device configured to bind the contralateral breast of the patient to the patient's body while pushing the contralateral breast away from treatment fields directed at a treatment breast of the patient, wherein the breast holder device is coupled to the treatment breast support.
